(19) United States
(12) Patent Application Publication
(10) Pub. No.: US 2023/0293454 A1
(43) Pub. Date: Sep. 21, 2023

(54) USE OF MITOXANTRONE HYDROCHLORIDE LIPOSOME AND PEGASPARGASE

(71) Applicant: CSPC ZHONGQI PHARMACEUTICAL TECHNOLOGY (SHIJIAZHUANG) CO., LTD, Shijiazhuang, Hebei (CN)

(72) Inventors: Chunlei LI, Shijiazhuang, Hebei (CN); Shujie YAN, Shijiazhuang, Hebei (CN); Yanhui LI, Shijiazhuang, Hebei (CN); Hongmei LIN, Shijiazhuang, Hebei (CN); Xuefang XIA, Shijiazhuang, Hebei (CN); Meng ZHOU, Shijiazhuang, Hebei (CN); Shixia WANG, Shijiazhuang, Hebei (CN); Shasha YANG, Shijiazhuang, Hebei (CN)

(73) Assignee: CSPC ZHONGQI PHARMACEUTICAL TECHNOLOGY (SHIJIAZHUANG) CO., LTD, Shijiazhuang, Hebei (CN)

(21) Appl. No.: 18/020,089

(22) PCT Filed: Aug. 6, 2021
(86) PCT No.: PCT/CN2021/111121

§ 371 Date: Feb. 7, 2023

(30) Foreign Application Priority Data
Aug. 7, 2020 (CN) 202010788006.5

(51) Int. Cl. A61K 31/136; A61K 38/50; A61K 9/127; A61P 35/00

(52) U.S. Cl. CPC A61K 31/136; A61K 38/50; A61K 9/127; A61P 35/00

(57) ABSTRACT

The present invention provides use of mitoxantrone hydrochloride liposome and pegaspargase in preparation of drug for treating NK/T-cell lymphoma (NKTCL). The NKTCL comprises initial-treating, relapsed, and refractory extranodal NKTCL. Preferably, the initial-treating, relapsed, and refractory extranodal NKTCL is initial-treating, relapsed, and refractory extranodal nasal NKTCL. On this basis, other first-line and second-line drugs for treating NKTCL can also be further used. The present invention further provides a method for treating NKTCL. The method relates to administering therapeutically effective amounts of mitoxantrone hydrochloride liposome and pegaspargase to a patient. The mitoxantrone hydrochloride liposome improves the curative effect of pegaspargase on NKTCL, has little toxic and side effects, and increases the complete response rate (CR rate) and the partial response rate (PR rate) of diseases.

### TECHNICAL FIELD

The present invention belongs to the field of antitumor, specifically relates to a use of mitoxantrone hydrochloride liposome and pegaspargase in the preparation of a drug for treating NK/T-cell lymphoma (NKTCL).

### BACKGROUND ART

NKTCL (NK/T-cell lymphoma) is clinically divided into nasal type (Extranodal NK/T-cell Lymphoma Nasal Type) and non-nasal type, and the nasal type is a rare type of aggressive non-Hodgkin lymphoma. The incidence of this disease is low, in proportion of about 11% to 14% of lymphoma in China (non-patent reference 1).

Although there are geographical differences in the incidence of NKTCL, the characteristics of this disease are similar in endemic areas such as Western countries and East Asia. It mostly occurs in the upper respiratory tract and digestive tract, about 80% of which occurs in the nasal cavity, and can also occur in organs outside the nasal cavity such as Waldeyer's ring and nasopharynx, and occasionally in the skin, digestive tract, testicles, etc. Although about 67% to 80% of patients are early-stage (stage I/II), the disease is strongly invasive and has a poor prognosis, and the 5-year overall survival (Over Survival, OS) rate is about 32%; The median OS is 8 months, and the 5-year OS rate of early-stage-NKTCL is 25% to 86%. Tumor pathogenesis is closely related to Epstein-Barr virus (EBV) infection, and quantitative detection of plasma EBV-DNA can reflect the tumor burden, progression and prognosis. The large variation in patient's survival is mainly due to disease heterogeneity and different choices of treatment strategies.

Most NKTCLs express mRNA of multi-drug resistance gene and its expression product P-glycoprotein (PgP), and produce multi-drug resistance (MDR), so that the CHOP or CHOP-like chemotherapy regimens based on anthracycline have the defects of low response rate, with complete response (CR) rate of 25% to 50%, 5-year OS rate of 0% to 34% (non-patent reference 2). Therefore, there is no literature reporting that mitoxantrone can be used for treating NKTCL, and the serious adverse reactions of mitoxantrone (bone marrow suppression, dose-limiting cardiotoxicity, gastrointestinal toxicity, and fatigue, etc.) limit its clinical use.

Liposome is a novel drug delivery system. Mitoxantrone liposomal preparations have been studied. For example, the Chinese patent application 200610102339.8 filed on Dec. 29, 2006 and the PCT application WO2008/080367A1 filed on Dec. 29, 2007, disclosed a mitoxantrone liposome, and the disclosure thereof is incorporated herein by reference. Studies have shown that the liposomal preparations have lower toxicity and better antitumor efficacy at lower doses compared with mitoxantrone free drug.

Antitumor drugs that are not affected by multi-drug resistance such as L-asparaginase (L-ASP), gemcitabine and the like are increasingly used in NKTCL, they have shown good efficacy whether in terms of CR rate, OS, or progression-free survival (PFS), and significant improvement of the prognosis of patients compared with CHOP or CHOP-like chemotherapy regimens. In particular, L-ASP can hydrolyze asparagine in serum, so that the syntheses of DNA, RNA and protein of tumor cells are inhibited, thereby playing a role of anti-tumor effect. Chemotherapy regimens containing L-ASP are effective in both patients in early diagnosed stage and patients in progressive, relapsed/refractory stages. The efficacy of L-ASP in NKTCL is significantly improved compared with anthracyclines, therefore, the chemotherapy containing L-ASP for NKTCL is significantly improved. For early NKTCL, the efficacy of the combination of radiotherapy+chemotherapy is higher than that of radiotherapy alone. The efficacy of the combination of the first course of radiotherapy followed by chemotherapy or the combination of the concurrent radiotherapy and chemotherapy is better than that of chemotherapy alone, with the 5-year OS rate 45% to 100%. A domestic study from two research centers reported that, comparing with the CHOP regimen for treating patients with early-stage (stage II.) NKTCL, the efficacy of LOP regimen (L-ASP+vincristine+dexamethasone) was significantly better. The CR rates of the two groups were 68.8% and 50% respectively, the 3-year OS rates were 87.5% and 62.5% respectively, and the PFS rates were 79.2% and 50% respectively (non-patent reference 3).

Since 2010, NCCN guidelines also recommend L-ASP-based combination chemotherapy as the first-line regimen for treating NKTCL. Although L-ASP-based chemotherapy regimens can significantly improve clinical efficacy and long-term survival, however, L-ASP is a heterologous protein derived from Escherichia coli that stimulates the body to produce antibodies, develop allergic reactions and life-threatening in severe cases, and reduces the efficacy of L-ASP and disease-free long-term survival.

In order to improve the efficacy and safety of L-ASP, Escherichia coli-derived L-ASP is linked to polyethylene glycol and lipid bilayer to form liposomal asparaginase PEG-ASP, so as to prolong the half-life, achieve low immunogenicity, significantly reduce allergic reactions, especially have less immediate allergic reactions, and other adverse reactions similar to L-ASP. Since 2013, PEG-ASP was recommended to replace L-ASP.

Domestic researchers applied a sandwich treatment regimen of pegaspargase, gemcitabine and oxaliplatin to newly diagnosed NKTCL patients, and it showed that a complete response (CR) rate was only 23.68%, partial response (PR) rate was 63.16%, and objective response rate (ORR) was 86.84% after at least 2 cycles (6 weeks) of treatment. After the completion of treatment, the ORR was 92.1% (CR rate was 86.84%, PR rate was 5.26%), 3-year OS rate and PFS rate were 72.6% and 57.8%, respectively, and grade 1/2 hematologic toxicity was common (non-patent references 4 and 5). Therefore, there is a need for improvements in combination chemotherapy regimens of pegaspargase.

However, due to the low incidence of NKTCL, there is a lack of large-scale randomized controlled studies on the treatment of the disease, and most of the current therapeutic evidence comes from retrospective analyses or early clinical trials (phase I/II) with small samples, which are still insufficient to form treatment recommendations with sufficient medical evidences. Therefore, it is difficult to improve the combination chemotherapy regimen of pegaspargase by referring to clinical data.

### LIST OF CITED DOCUMENTS

### Non-Patent References

non-patent reference 1: Li Y X, et al. Radiotherapy alone with curative intent in patients with stage I extranodal nasal-type NK/T-cell lymphoma. Int J Radiat Oncol Biol Phys. 2012 Apr. 1; 82(5):1809-1815.
non-patent reference 2: Isobe K, Uno T, Tamaru J, et al. Extranodal natural killer/T-cell lymphoma, nasal type: The significance of radiotherapeutic parameters. Cancer, 2006, 106: 609-615.
non-patent reference 3: Hamaguchi M, et al. Phase II study of SMILE chemotherapy for newly diagnosed stage IV, relapsed, or refractory extranodal natural killer (NK)/T-cell lymphoma, nasal type: the NK-Cell Tumor Study Group study. J Clin Oncol. 2011 Nov. 20; 29(33):4410-4416.
non-patent reference 4: Jing X M, et al. Efficacy and tolerance of gemcitabine and oxaliplatin with sandwiched radio-therapy in the treatment of newly-diagnosed extra nodal nature (NK)/Tcell lymphoma. LeukRes. 2016, 47:26-31.
non-patent reference 5: Wang J H, et al, Analysis of the efficacy and safety of a combined gemcitabine, oxaliplatin and pegaspargase regimen for NK/T-cell lymphoma. Oncotarget. 2016 Jun. 7; 7(23):35412-35422.

### SUMMARY OF THE INVENTION

Unless otherwise specified, the terms of “treatment naïve”, “relapsed”, “refractory”, “asparaginase/pegaspargase-containing chemotherapy regimen” in the present invention are defined as follows:

“Treatment naïve” is defined as being diagnosed for the first time, and has not been treated.

“Relapsed” is defined as the occurrence of new lesions at the primary site or other sites after the complete response (CR) is achieved with administrating asparaginase/pegaspargase-containing chemotherapy regimen.

“Refractory” is defined as any of the following situations: the efficacy evaluation is progressive disease (PD) after 2 cycles of treatment with applying asparaginase/pegaspargase-containing chemotherapy regimen, or the efficacy evaluation do not reach partial response (PR) after 4 cycles of treatment, or the efficacy evaluation do not reach complete response (CR) after 6 cycles of treatment.

“Asparaginase/pegaspargase-containing chemotherapy regimen” refers to the combination chemotherapy regimen of asparaginase or pegaspargase described in the background technique, such as SMILE regimen, GEMOX regimen, AspaMet-Dex regimen or LOP regimen.

The present invention provides a use of mitoxantrone liposome and pegaspargase in the preparation of a drug for treating NKTCL.

At the same time, the present invention provides a use of mitoxantrone liposome in the preparation of drugs improving the efficacy of pegaspargase in the treatment of NKTCL.

Further, the present invention provides a drug for treating NKTCL, which is characterized in that it comprises mitoxantrone liposome and pegaspargase.

Preferably, the mitoxantrone liposome of the present invention is preferably mitoxantrone hydrochloride liposome.

The NKTCL in the present invention includes treatment naïve, relapsed, refractory extranodal NKTCL. Preferably, the treatment naïve, relapsed, refractory NKTCL is treatment naïve, relapsed, refractory extranodal nasal NK/T-cell lymphoma.

The drugs may further include other first-line and second-line drugs for treating NKTCL, and the drugs are the first-line and second-line drugs approved by drug regulatory authorities in China or other countries and regions (such as the United States, the European Union, Japan, South Korea, etc.) for treating NKTCL.

Preferably, the drug may be an injection dosage form, including liquid injection, powder for injection, tablet for injection and the like. Mitoxantrone hydrochloride liposome and pegaspargase may be present in the same preparation or in separated preparations. When the mitoxantrone hydrochloride liposome is used as a liquid injection, calculated on the basis of mitoxantrone, it contains 0.5 to 5 mg/ml active ingredient, preferably 1 to 2 mg/ml, more preferably 1 mg/ml. When pegaspargase is an injection, it contains asparaginase in an amount of 1000-5000 IU/5 ml, preferably asparaginase 3750 IU/5 ml.

The present invention provides a method for treating NKTCL, wherein: therapeutically effective amounts of mitoxantrone hydrochloride liposome and pegaspargase are administered to a NKTCL patient. The administration is preferably given by injection.

The present invention also provides a method for improving the efficacy of pegaspargase for treating NKTCL, wherein: on the basis of the administration of pegaspargase to NKTCL patients, it further comprises an administration of a therapeutically effective amount of mitoxantrone hydrochloride liposome in combination.

Preferably, calculated on the basis of mitoxantrone, the therapeutically effective amount of mitoxantrone hydrochloride liposome is 8 to 30 mg/m², more preferably 12 to 24 mg/m². For example, 12 mg/m², 14 mg/m², 16 mg/m², 18 mg/m², 20 mg/m², 24 mg/m². Preferably, the administration mode of mitoxantrone hydrochloride liposome is administered intravenously. Preferably, the administration cycle is administered once every 3 weeks. The administration is no more than 6 cycles at most. Preferably, for intravenous administration every time, the infusion time of the liposomal pharmaceutical preparation is 30 min to 120 min, preferably 60 min to 120 min, further preferably 90±15 min.

Calculated on the basis of asparaginase, the dose of pegaspargase is 2000 to 2500 IU/m², preferably in intramuscular injection, and it can be administered at any time before, during and after the administration of mitoxantrone liposome. The administration cycle is the same as that of mitoxantrone hydrochloride liposome.

The present invention further provides a composition for treating NKTCL, comprising mitoxantrone hydrochloride liposome and pegaspargase, wherein a therapeutically effective amount of mitoxantrone hydrochloride liposome is administered to NKTCL patients, and 2000˜2500 IU/m² of pegaspargase is administered at any time before, during, and after the administration of mitoxantrone liposome.

The present invention further provides a drug for improving the efficacy of pegaspargase in the treatment of NKTCL, wherein the drug comprises mitoxantrone hydrochloride liposome, and the mitoxantrone liposome is administered at any time before, during, and after the administration of pegaspargase, at a dose of 8 to 30 mg/m², administered once every 3 weeks.

The dose of mitoxantrone hydrochloride liposome of the present invention is calculated on the basis of mitoxantrone.

The mitoxantrone hydrochloride liposome described in the present invention can be prepared by conventional methods in the art, and the mitoxantrone hydrochloride liposome can be prepared by any method disclosed in the prior art, such as by the method disclosed in WO2008/080367 A1, the entire contents of which are incorporated herein as a reference.

In some embodiments, the mitoxantrone hydrochloride liposome of the present invention, with a particle size of about 30 to 80 nm, comprises: 1) the active ingredient mitoxantrone, which can form an insoluble precipitation with multivalent counterion in liposome, 2) lipid bilayer containing phospholipid with a phase transition temperature (Tm) higher than the body temperature, so that the phase transition temperature of the liposome is higher than the body temperature. The phospholipid with the Tm higher than the body temperature is phosphatidylcholine, hydrogenated soy lecithin, hydrogenated ovolecithin, lecithin bis palmitate or lecithin bis stearate or any combination thereof, and the particle size is about 35 to 75 nm, preferably 40 to 70 nm, further preferably 40 to 60 nm, especially preferably 60 nm. The lipid bilayer contains hydrogenated soy lecithin, cholesterol and distearoylphosphatidylethanolamine modified with polyethylene glycol 2000 in a mass ratio of 3:1:1, the particle size is about 60 nm, and the counterion is sulfate ion. Alternatively, the lipid bilayer of the liposome contains hydrogenated soy lecithin, cholesterol and distearoylphosphatidylethanolamine modified with polyethylene glycol 2000 in a mass ratio of 3:1:1, the particle size is about 40 to 60 nm, the counterion is sulfate ion, further, the weight ratio of hydrogenated soy lecithin:cholesterol:distearoylphosphatidylethanolamine modified with polyethylene glycol 2000:mitoxantrone is 9.58:3.19:3.19:19:1.

In some embodiments, the mitoxantrone hydrochloride liposome of the present invention are prepared as follows: HSPC (hydrogenated soy lecithin), Chol (cholesterol) and DSPE-PEG2000 (di stearoylphosphatidylethanolamine modified with polyethylene glycol 2000) are weighed in a mass ratio of (3:1:1), dissolved in 95% ethanol to obtain a clear solution (i.e., ethanol solution of phospholipid). Ethanol solution of phospholipid is mixed with 300 mM ammonium sulfate solution and the resultant is hydrated for 1 hour by shaking at 60 to 65° C. to obtain heterogeneous multilamelar liposome. The microfluidic equipment was then used to reduce the particle size of liposome. After the obtained sample was diluted 200-fold with NaCl solution in a concentration of 0.9%, it was detected with NanoZS, and the average particle size of the particles was about 60 nm, with the main peak concentrated between 40 to 60 nm. Then, the ultrafiltration device was used to remove the ammonium sulfate of the blank liposome external phase, and the external phase was replaced with 290 mM sucrose and 10 mM glycine to form an ammonium sulfate transmembrane gradient. Based on a ratio of liposome to drug of 16:1, mitoxantrone hydrochloride solution (10 mg/mL) was added to the blank liposome, and the drug was loaded at 60 to 65° C. After incubated for approximately 1 hour, the encapsulation efficiency of approximately 100% can be demonstrated using gel exclusion chromatography. The product obtained from this formulation is named as PLM 60. The weight ratio of HSPC:Chol:DSPE-PEG2000:mitoxantrone in PLM60 is 9.58:3.19:3.19:1, and the osmotic pressure of sucrose-glycine solution is close to the physiological value.

### Beneficial Technical Effects

By administering mitoxantrone hydrochloride liposome and pegaspargase in combination to a NKTCL patient, the efficacy of pegaspargase for treating NKTCL in treatment naïve patients and relapsed or refractory patients can be improved, the disease remission rate can be improved, and it has a favorable safety and tolerability, with less toxicity and side effects, especially the complete response rate (CR rate) and partial response rate (PR rate) of the disease are improved, and the disease progression can be controlled.

### Specific Embodiment

The following embodiments are specific descriptions of the present invention and should not limit the scope of the present invention.

### EXAMPLE 1 CLINICAL STUDY OF MITOXANTRONE HYDROCHLORIDE LIPOSOME INJECTION FOR TREATING TREATMENT NAÏVE AND RELAPSED/REFRACTORY NKTCL

This was a single-arm, open-label, multicenter phase I/II clinical study in which subjects with treatment naïve or relapsed/refractory extranodal nasal NK/T-cell lymphoma were assigned to receive different doses of mitoxantrone hydrochloride liposome injection and fixed-dose pegaspargase injection, in order to research the safety and tolerability of the combination regimen, determine the optimal dose of mitoxantrone hydrochloride liposomal injection in the combination regimen, and evaluate the efficacy and observe the pharmacokinetic characteristics.

The study was divided into a dose-escalation phase and a dose-expansion phase.

I. Design of Experiments

1. Dose-Escalation Phase

(1) Study Design

The study included a screening period, a treatment period, and a follow-up period.

After signing the informed consent form and completing all baseline tests during the screening period, eligible subjects entered the treatment period and were treated by administrating a combination of mitoxantrone hydrochloride liposome injection with pegaspargase injection, and the administration dose of mitoxantrone hydrochloride liposome injection was gradually increased from the low-dose group to the high-dose group, and the administration dose for the subject was the dose of the drug that was being used or would be used at the time of entering this study. The administration regimen was carried out once every 3 weeks, the first cycle of the treatment period was the DLT (dose-limiting toxicity) observation period, and the subject who had completed 3 weeks of treatment and observation could continue the next cycle of administration until progression, death, withdrawal from the study as required by the subject or his/her legal representative, starting on another new treatment, and the end of the entire study (whichever occurred first), and the administration was no more than 6 cycles at most. Efficacy evaluations were performed every 2 cycles (6 weeks) during treatment. All subjects were required to collect PK (pharmacokinetic) blood samples at different time points before and after administration as prescribed by the protocol, and relevant tests were refined during the study to observe safety and tolerability. An end-of-treatment visit was performed 4 weeks after the last administration. After the end of the treatment, the follow-up period would be entered, and progression-free survival (PFS) follow-up was conducted once every 8 weeks in the subjects who had received at least one administration dose, and 5-year overall survival (OS) rates follow-up was conducted every 8 weeks in the subjects who had already progressed and started other new treatment.

(2) Dose Escalation Regimen

Mitoxantrone hydrochloride liposome: 12 mg/m² was used as the initial dose (based on mitoxantrone), and 4 dose groups in total of 12 mg/m², 16 mg/m², 20 mg/m², 24 mg/m² were designed.

Subjects with DLT were treated by investigators in accordance with clinical criterion for diagnosis and treatment, and could continue the next cycle of administration by delaying administration for no more than 2 weeks, maintaining the original dose, or reducing one dose level. The administration dose was not allowed to increase for the same subject.

Pegaspargase injection: i.e., liposomal asparaginase (PEG-ASP), the administration dose was 2000 IU/m², for intramuscular administration, which could be administered at any time before, during, and after the administration of mitoxantrone hydrochloride liposome.

(3) Extended Administration after Completion of the Observation Period of DLT

Subjects with DLT were treated by investigators in accordance with clinical criterion for diagnosis and treatment, and could continue the next cycle of administration by delaying administration for no more than 2 weeks, maintaining the original dose, or reducing one dose level. The administration dose was not allowed to increase for the same subject.

If MTD (maximum tolerated dose) of mitoxantrone hydrochloride liposome injection was found, MTD was selected as RP2D (phase II recommended dose); If the MTD of mitoxantrone hydrochloride liposome injection was not found, RP2D was determined by the investigator and sponsor in consultation based on the results of the DLT observation period in the dose escalation phase. The RP2D dose was selected for expansion during the dose expansion phase.

2. Dose Expansion Phase

The extended study was conducted in two groups at the RP2D level: the first group included newly treated extranodal nasal type NK/T cell lymphoma, and the second group included relapsed/refractory extranodal NK/T cell lymphoma, and both groups were treated with mitoxantrone hydrochloride liposome injection combined with pegaspargase injection, and in each group 10 to 40 effective cases were expanded to explore the efficacy and tolerability of the combination regimen.

The study included a screening period, a treatment period, and a follow-up period.

After signing the informed consent form and completing all baseline tests during the screening period, eligible subjects entered the treatment phase. The administration regimen was carried out once every 3 weeks, until disease progression, death, withdrawal from the study as required by the subject or his/her legal representative, starting on another new treatment, and the end of the entire study (whichever occurred first), and the administration was no more than 6 cycles at most. Efficacy evaluations were performed every 2 cycles (6 weeks) during treatment. All subjects were required to collect PK (pharmacokinetic) blood samples at different time points before and after administration as prescribed by the protocol, and relevant tests were refined during the study to observe safety and tolerability. An end-of-treatment visit was performed 4 weeks after the last administration. After the end of the treatment, the follow-up period would be entered, and progression-free survival (PFS) follow-up was conducted once every 8 weeks in the subjects who had received at least one administration dose, and 5-year overall survival (OS) rates follow-up was conducted every 8 weeks in the subjects who had already progressed and started other new treatment.

3. Study End Time

Dose escalation phase: Study ended after RP2D was determined.

Dose expansion phase: The study ended after CR rate (complete response rate), PR rate (partial response rate), ORR (objective response rate), DCR (disease control rate), incidence rate of adverse events during treatment and safety follow-up period, vital signs, physical examination, laboratory test abnormalities, etc. were observed.

II. Study Population

(1) Inclusion Criteria

Subjects who met all the following inclusion criteria before enrollment:
1. Subject fully understand and voluntarily participate in this study and sign informed consent.
2. Age≥18, ≤75 years.
3. Histologically confirmed diagnosis of treatment-naive, relapsed or refractory extranodal NK/T-cell lymphoma nasal type (NKTCL) Treatment naïve.
Relapsed NKTCL was defined by the appearance of any new lesion at the primary site or at other sites after treatment with asparaginase/pegaspargase-containing chemotherapy regimen to achieve complete response (CR).
Refractory NKTCL was defined as any one of the following: the efficacy evaluation was progressive disease (PD) after two cycles (6 weeks) of treatment with asparaginase/pegaspargase-containing chemotherapy regimen, or the efficacy evaluation failure to achieve partial response (PR) after 4 cycles (12 weeks), or the efficacy evaluation failure to achieve complete response (CR) after 6 cycles (18 weeks).
4. ECOG (Eastern Cooperative Oncology Group) score 0˜1;
5. At least one measurable lesion as per Lugano 2014 criteria.
6. The organ function level must meet the following requirements:
   (1) Bone marrow: absolute neutrophil count (ANC)≥1.5×10⁹/L, hemoglobin≥80 g/L (no transfusion within 14 days), platelet≥75×10⁹/L;
   (2) liver function: aspartate aminotransferase (AST), alanine aminotransferase (ALT)≤3 times the upper limit of normal (ULN), total bilirubin (TBIL)≤1.5×ULN (AST, ALT≤5×ULN if documented hepatic involvement of lymphoma);
   (3) Renal function: serum creatinine≤1.5×ULN;
   (4) Coagulation function: International normalized ratio (INR) and activated partial thromboplastin time (APTT)≤1.5×ULN.

(II) Exclusion Criteria

Subjects Who Met any of the Following Criteria were not Eligible for Inclusion in this Study

1. For NKTCL any of the following situations occurred:
(1) Known infiltration of the bone marrow according to criteria for leukemia (20% myeloblast);
(2) Lymphoma central invasion;
(3) Hemophagocyic syndrome.

2. History of allergy and contraindications to mitoxantrone hydrochloride and/or asparaginase/pegaspargase.

3. Chemotherapy, immunotherapy, targeted therapy, endocrine therapy, and radiotherapy within 4 weeks of the first dose of mitoxantrone hydrochloride liposome injection (2 weeks for the local radiation therapy for pain relief).

4. Life expectancy<3 months.

5. For heart function any of the following conditions occurred:
(1) Impaired cardiac function or serious heart disease: myocardial infarction, congestive heart failure, heart disease with symptoms requiring drug treatment such as unstable angina and arrhythmia, uncontrolled severe hypertension within 6 months before screening, viral myocarditis or history of persistent cardiomyopathy within 6 months;
(2) Cardiac function grades III˜IV;
(3) Left ventricular ejection fraction<50% on echocardiography;
(4) In the absence of a pacemaker, degree II/III of atrioventricular block, long QT syndrome, or QTc>480 ms was present on 12-lead ECG.

6. Infectious disease screening: hepatitis B surface antigen was positive and HBV-DNA titer was above the upper limit of the study center, hepatitis C virus antibody was positive and HCV-RNA titer was above the upper limit of the study center, human immunodeficiency virus (HIV) antibody was positive in initial screening.

7. Acute symptomatic or chronic pancreatitis within 4 weeks prior to screening.

8. History of solid organ transplantation, autologous hematopoietic stem cell transplantation within 6 months prior to screening, or allogeneic hematopoietic stem cell transplantation before screening.

9. History of, or known additional tumor (exception: non-melanoma skin cancer (in situ) and cervical cancer (in situ) which had been cured and have not recurred within 5 years).

10. Major surgery within 4 weeks prior to screening or had a surgical schedule during the study period.

11. A serious infection within 4 weeks prior to screening and not suitable for the study according to the judgment of the investigator.

12. Uncontrolled diabetes at screening.

13. History of active bleeding within 3 months prior to screening.

14. Known drug abuse (use of narcotic drugs or psychotropic drugs for non-medical purposes) or drug dependence (sedative-hypnotics, analgesics, narcotics, stimulants, psychotomimetic drugs, etc.).

15. Known psychiatric disorders or cognition disorders.

16. Pregnant or breastfeeding women, or patients who were expecting to conceive or father in 12 months

17. Not suitable for this study as determined by the investigator due to other reasons.

(III) Discontinuation/Termination Criteria

Subjects who met any of the follow criteria during the study would be withdrawn from the study.

1. Subject declined further study participation.

2. Subject was non-compliant with protocol based on investigator and/or sponsor's assessment.

3. Any adverse events which, in the opinion or the investigator, indicated that continued participation in the study would increase the risk for the subject.

4. Subjects had disease progression after administration.

5. Pregnancy.

III. Results of the Study

Disease assessment was performed utilizing PET-CT, tumor responses were assessed according to Lugano 2014 criteria.

A total of 24 subjects were enrolled in this study, including 15 subjects with treatment naïve treatment-naive extranodal NK/T-cell lymphoma (nasal type) and 9 subjects with relapsed/refractory extranodal NK/T-cell lymphoma (nasal type), of which subjects who underwent one efficacy evaluation (6 weeks) were enrolled for the following assessment:

Group I: 11 subjects with treatment naïve treatment-naive extranodal NK/T-cell lymphoma (nasal type).

Group II: 9 subjects with relapsed/refractory extranodal NK/T-cell lymphoma (nasal type).

The dosage of mitoxantrone hydrochloride liposome was shown in Table 1 below, and the dosage of pegaspargase was 2000 IU/m².

The results of the evaluation were shown in Table 1 below:

TABLE 1

| | Treatment-naïve treatment naïve subjects | | | | | Relapsed/refractory subjects | | | | |
|---|---|---|---|---|---|---|---|---|---|---|
| | 12 | 16 | 20 | 24 | | 12 | 16 | 20 | 24 | |
| Dosage | mg/m² | mg/m² | mg/m² | mg/m² | Total | mg/m² | mg/m² | mg/m² | mg/m² | Total |
| CR (complete | 2 | 2 | 4 | 1 | 9 | 0 | 1 | 2 | 0 | 3 |
| response) | | | | | | | | | | |
| PR (partial | 0 | 0 | 0 | 2 | 2 | 0 | 2 | 0 | 2 | 4 |
| response) | | | | | | | | | | |
| SD (Stable | 0 | 0 | 0 | 0 | 0 | 1 | 1 | 0 | 0 | 2 |
| Disease) | | | | | | | | | | |
| PD (Progressive | 0 | 0 | 0 | 0 | 0 | 0 | 0 | 0 | 0 | 0 |
| Disease) | | | | | | | | | | |

| | | |
|---|---|---|
| CR rate | 81.8% (9/11) | 33.3% (3/9) |
| PR rate | 18.2% (2/11) | 44.4% (4/9) |
| ORR (Objective | 100% (11/11) | 77.8% (7/9) |
| Response Rate) | | |
| DCR (Disease | 100% (11/11) | 100% (9/9) |
| Control Rate) | | |

Results: The treatment of mitoxantrone hydrochloride liposome combined with pegaspargase was safe and well tolerated in both treatment-naïve subjects and relapse/refractory subjects group, with less toxicities and significant curative effects, in particular, the CR rate and PR rate of different subjects can be improved, and the disease control rate was 100%, and there was no case of disease progression during treatment. It indicated that the administration of mitoxantrone hydrochloride liposome combined with pegaspargase had broad clinical application prospects in the treatment of NKTCL.

The foregoing is only a specific embodiment of the present invention, but the scope of protection of the present invention is not limited thereto, and any change or replacement which is easily conceived of by a person skilled in the art who is familiar with the art should belong to the scope of the technology disclosed in the present invention, should be covered within the scope of protection of the present invention. Therefore, the scope of protection of the present invention shall prevail the scope of protection of the claims.

Practicability

The pharmaceutical composition provided according to the present invention may be used in the field of antitumor, in particular for the treatment of NKTCL.

1. A use of mitoxantrone liposome and pegaspargase in the preparation of a drug for treating NK/T-cell lymphoma.

2. A use of mitoxantrone liposome in the preparation of a drug for improving the efficacy of pegaspargase for treating NK/T-cell lymphoma.

3. The use according to claim 1, wherein the NK/T-cell lymphoma includes treatment naïve, relapsed, refractory extranodal NK/T-cell lymphoma; the treatment naïve, relapsed, refractory extranodal NK/T-cell lymphoma is treatment naïve, relapsed, refractory extranodal nasal NK/T-cell lymphoma; and the mitoxantrone liposome is mitoxantrone hydrochloride liposome.

4. The use according to claim 3, wherein the drug is an injection dosage form, including liquid injection, powder for injection, tablet for injection; mitoxantrone hydrochloride liposome and pegaspargase are present in the same preparation, or in separated preparations; when the mitoxantrone hydrochloride liposome is liquid injection, calculated on the basis of mitoxantrone, it contains 0.5 to 5 mg/ml active ingredient; when asparaginase is a liquid injection, it contains asparaginase in an amount of 1000 to 5000 IU/5 ml.

5. The use according to claim 1, wherein the drug further comprises other drug for treating NK/T-cell lymphoma.

6. A drug for treating NK/T-cell lymphoma, wherein the drug comprises mitoxantrone hydrochloride liposome and pegaspargase, the drug is an injection dosage form, including liquid injection, powder for injection, tablet for injection; mitoxantrone hydrochloride liposome and pegaspargase are liquid injection; calculated on the basis of mitoxantrone, mitoxantrone hydrochloride liposome contains active ingredient in an amount of 0.5 to 5 mg/ml; pegaspargase contains asparaginase in an amount of 1000 to 5000 IU/5 ml; mitoxantrone hydrochloride liposome and pegaspargase are present in the same preparation or in separated preparations.

7. A method for treating NK/T-cell lymphoma, wherein: therapeutically effective amounts of mitoxantrone hydrochloride liposome and pegaspargase are administered to a patient with NK/T-cell lymphoma, and the administration is injection form; calculated on the basis of mitoxantrone, the therapeutically effective amount of mitoxantrone hydrochloride liposome is 8 to 30 mg/m², and the administration cycle is once every 3 weeks; and the dosage of pegaspargase is 2000 to 2500 IU/m², administered intramuscularly.

8. A method for improving the efficacy of pegaspargase on treating NK/T-cell lymphoma, wherein: on the basis of the administration of pegaspargase to a patient with NK/T cell lymphoma, it further comprises an administration of a therapeutically effective amount of mitoxantrone hydrochloride liposome in combination; based on mitoxantrone, the therapeutically effective amount of mitoxantrone hydrochloride liposome is 8 to 30 mg/m², and the administration cycle is once every 3 weeks; the dosage of pegaspargase is 2000 to 2500 IU/m², intramuscular administration.

9. A composition for treating NK/T-cell lymphoma, comprising mitoxantrone hydrochloride liposome and pegaspargase, wherein: mitoxantrone hydrochloride liposome is administered in an amount of 8 to 30 mg/m² to a patient with NK/T-cell lymphoma, and the administration cycle is once every 3 weeks; and pegaspargase is administered in an amount of 2000 to 2500 IU/m² at any time before, during and after the administration of mitoxantrone liposome.

10. A drug for improving the efficacy of pegaspargase for treating extranodal NK/T-cell lymphoma, wherein: the drug contains mitoxantrone hydrochloride liposome, the mitoxantrone liposome is administered at any time before, during and after the administration of pegaspargase, at a dosage of 8-30 mg/m², administered once every 3 weeks.

11. The use according to claim 1, wherein the particle size of mitoxantrone hydrochloride liposome is about 30 to 80 nm, and mitoxantrone hydrochloride liposome contains: 1) the active ingredient mitoxantrone, 2) lipid bilayer containing phospholipid with a phase transition temperature (Tm) higher than body temperature, wherein the phospholipid with the Tm higher than the body temperature is phosphatidylcholine, hydrogenated soy lecithin, hydrogenated ovolecithin, lecithin bis palmitate, lecithin bis stearate and any combination thereof.

12. The drug according to claim 6, wherein the particle size of mitoxantrone hydrochloride liposome is about 30 to 80 nm, and mitoxantrone hydrochloride liposome contains: 1) the active ingredient mitoxantrone, 2) lipid bilayer containing phospholipid with a phase transition temperature (Tm) higher than body temperature, and the phospholipid with the Tm higher than body temperature is phosphatidylcholine, hydrogenated soy lecithin, hydrogenated ovolecithin, lecithin bis palmitate, lecithin bis stearate and any combination thereof.

13. The method according to claim 7, wherein the particle size of mitoxantrone hydrochloride liposomal is about 30 to 80 nm, and mitoxantrone hydrochloride liposome contains: 1) the active ingredient mitoxantrone, 2) lipid molecular bilayer contains phospholipid with a phase transition temperature (Tm) higher than body temperature, the phospholipid with the Tm higher than the body temperature is phosphatidylcholine, hydrogenated soy lecithin, hydrogenated ovolecithin, lecithin bis palmitate, lecithin bis stearate and any combination thereof.
